(19) United States
(12) Patent Application Publication
(10) Pub. No.: US 2018/0042551 A1
(43) Pub. Date: Feb. 15, 2018

(54) DEVICE IN THE FORM OF A GARMENT FOR MONITORING A PHYSIOLOGICAL PARAMETER OF A USER

(71) Applicant: Bioserenity, Paris (FR)

(72) Inventors: Marion Gouthez, Saint-Genis-Laval (FR); Sylvain Zorman, Paris (FR); Pierre-Yves Frouin, Paris (FR)

(73) Assignee: Bioserenity, Paris (FR)

(21) Appl. No.: 15/554,522

(22) PCT Filed: Mar. 4, 2016
(86) PCT No.: PCT/EP2016/054706

§ 371 Date: Aug. 30, 2017

(30) Foreign Application Priority Data
Mar. 6, 2015 (FR) 1551896

(51) Int. Cl. A61B 5/00; D04B 1/12; A61B 5/087; D04B 1/24; A61B 5/113; A61B 5/08

(52) U.S. Cl. CPC A61B 5/6805; A61B 5/1135; A61B 5/4866; A61B 5/082; A61B 5/087; A61B 5/0816; D04B 1/246; D04B 1/12

(57) ABSTRACT

The invention relates to a device for monitoring the breathing of a user comprising:
a textile support comprising a tubular portion formed by knitting an electrically insulating majority ground yarn, the tubular portion being able to cover the chest of the user,
at least one breathing sensor formed by knitting a detection yarn, the detection yarn comprising an internal core made of an electrically insulating material and an external sheath surrounding the internal core, the external sheath being formed made of an electrically conductive material,
wherein the breathing sensor forms a conductive band having a first end and a second end positioned at a distance from each other, the ends being able to be connected to an apparatus for measuring the electric resistance of the conductive band.

### FIELD OF THE INVENTION

The invention relates to a device for monitoring breathing, as a clothing item which may be worn by the user.

### STATE OF THE ART

In order to continuously monitor the physiological condition of a user, it is known to fix sensors in textile structures intended to form a clothing item. Such sensors for example allow measurement of an electrocardiographic signal which is representative of the heart activity of a user who wears the clothing item.

However, such sensors may sometimes prove to be bulky, causing consequently a lack of comfort for the user. Further, the assembling of these sensors on the clothing item and their electric connection may prove to be complex.

Document EP 1 506 738 describes an elastic clothing item comprising textile sensors. The sensors include flexible textile electrodes applied against the skin used for collecting electric signals generated by the body for the elaboration of an electrocardiogram or an electromyogram on the one hand, and a mechanical deformation sensor used for measuring the respiratory movements of the user on the other hand. The electrodes are formed by weaving or knitting an elastic conductive yarn. The mechanical deformation sensor comprises a non-knitted elastic conductive yarn. The elastic conductive yarn is obtained by wrapping a non-extensible conductive yarn around a core consisting of non-conductive elastic yarn, i.e. the conductive yarn is helically wound around the non-conductive elastic yarn. When the elastic conductive yarn is stretched, the yarn elongates and the neighbouring turns of the winding move away from each other, causing a measurable change in electric resistance which depends on the elongation of the elastic conductive yarn.

Document US 2007/0171024 describes a clothing item in which is integrated a gauge giving the possibility of monitoring the breathing of the user who wears the clothing item. The clothing item comprises a textile base woven from non-conductive yarns. The gauge is formed by a conductive yarn gimped with a non-conductive yarn, and woven through the textile base, at the abdominal region or at the breast of the user. The conductive yarn consists of ultra-fine metal yarns twisted with textile fibres or textile fibres mixed with metal fibres. The respiratory movements of the user cause an elongation or a contraction of the conductive yarn. The change in the length of the conductive yarn causes a change in the electric properties of the yarn.

In such a clothing item, the connection of the conductive yarn to a measuring apparatus requires first stripping the conductive yarn, i.e. removing the non-conductive yarn which surrounds it, in order to be able to make an electric contact between the conductive yarn and a connecting cable.

Further, the wrapping of the conductive yarn with a non-conductive yarn increases the total diameter of the conductive yarn, which increases the costs for making the clothing item and may generate discomfort for the user who wears the clothing item.

### SUMMARY OF THE INVENTION

An object of the invention is to propose a device for tracking the breathing of a user, which both has improved comfort and which minimizes the manufacturing steps required for making the device.

This object is attained within the scope of the present invention by means of a device for monitoring breathing of a user comprising:
a textile support comprising a tubular portion formed by knitting an electrically insulating majority ground yarn, the tubular portion being able to cover the chest of the user,
at least one breathing sensor formed by knitting a detection yarn, the detection yarn forming a plurality of stiches, the detection yarn comprising at least one internal core in an electrically insulating material and an external sheath surrounding the internal core, the external sheath being formed in an electrically conducting material so as to generate electric contacts between the stitches of the detection yarn.

The breathing sensor forms a conductive band having a first end and a second end positioned at a distance from each other, the ends being able to be connected to an apparatus for measuring the electric resistance of the conductive band.

The conductive band is positioned relatively to the tubular portion so that when the chest of the user is covered with the textile support, the conductive band is stretched and shrunk alternately because of the breathing of the user, the stretching and the shrinking of the conductive band having the effect of modifying the electric contacts between the stitches of the detection yarn within the conductive band, causing modification of the electric resistance of the conductive band.

In such a device, the detection yarn is not insulated, which allows the use of a smaller yarn, less costly and more lightweight. In addition, the connecting of the detection yarn to the measuring apparatus does not require any preliminary stripping of the detection yarn.

Further, the successive stitches of the detection yarn form multiple contacts of the external sheath with itself. It is mainly the modification of these electric contacts during the stretching and the shrinking of the conductive band which generates a modification of the electric resistance, and not a stretching or contraction of the actual yarn.

The proposed device may further have the following features:
the detection yarn comprises at least one core made of a polymer material, preferably of polyamide,
according to a first possibility, the sheath of the detection yarn is formed by wrapping the internal core with a yarn made of a conducting material, preferably of silver,
according to a second possibility, the sheath of the detection yarn is formed by coating the internal core with a layer made of a conductive material, preferably of silver,
the yarn may consist of several non-conductive filaments, each filament being coated with an external layer made of a conductive material, preferably of silver,
in this case, the coated filaments may be twisted together,
the tubular portion is formed by knitting the majority ground yarn and an elastic yarn,
the conductive band surrounds at least partly the chest of the user, preferably at the height of the sternum and/or the abdominal muscles, and extends over the belly and/or over the back of the user,
the conductive band may extend both over the belly and over the back of the user,
the tubular portion and the breathing sensor are formed by circular knitting in a single operation, the ground yarn and the detection yarn being alternately knitted,
during the knitting, the detection yarn is cut when the ground yarn is knitted,
the device further comprises an insulating layer positioned between the conductive band and the skin of the user when the user is covered with the textile support,
the device further comprises a sheath attached on the tubular portion and connecting cables located inside the sheath for connecting the ends of the conductive band to the apparatus for measuring electric resistance.

### PRESENTATION OF THE DRAWINGS

Other features and advantages will further become apparent from the description which follows, which is purely illustrative and non-limiting and should be read with reference to the appended figures, wherein:

FIG. 1 schematically illustrates a front view of a device for monitoring breathing according to a possible embodiment of the invention,

FIG. 2 schematically illustrates a rear view of the device for monitoring breathing,

FIG. 3 is a detailed view of the device,

FIG. 4 schematically illustrates the device including a sheath and connecting cables,

FIGS. 5A and 5B are schematic detailed views respectively of the face and of the back of the device,

FIGS. 6 and 7 schematically illustrate stitches forming a breathing sensor, when the sensor is in the rest condition and in the stretched condition respectively,

FIGS. 8 and 9 schematically illustrate a conductive yarn forming stitches, when the conductive yarn is in the rest condition and in the stretched condition respectively,

FIG. 10 schematically illustrates the structure of a non-conductive yarn used for forming the textile support,

FIG. 11 schematically illustrates the structure of a conductive yarn used for forming the sensor according to a first possibility,

FIG. 12 schematically illustrates the structure of a conductive yarn used for forming the sensor according to a second possibility,

FIG. 13 schematically illustrates the structure of a conductive yarn used for forming the sensor according to a third possibility,

FIG. 14 is an equivalent electric diagram of the breathing sensor and of an apparatus for measuring the electric resistance of the conductive band,

FIG. 15 schematically illustrates variations of electric resistance recorded from the breathing sensor.

### DETAILED DESCRIPTION OF AN EMBODIMENT

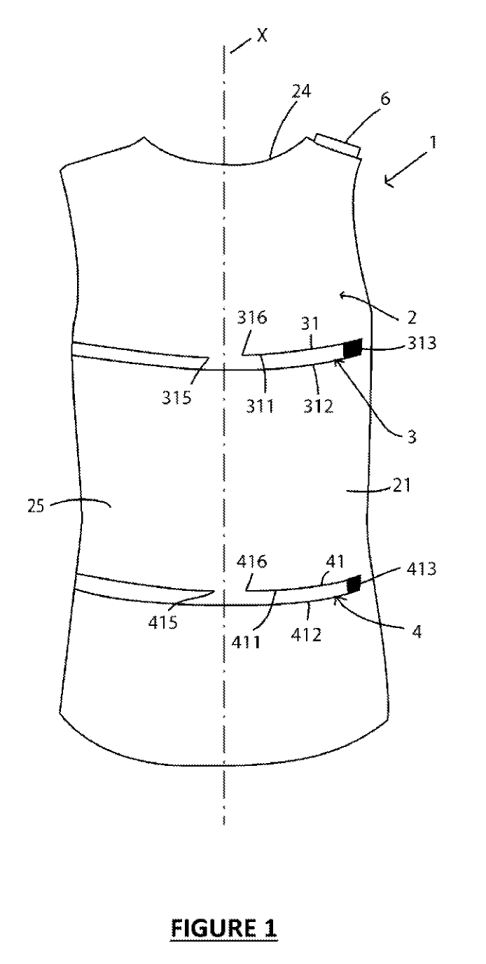

In FIGS. 1 to 4, the device for monitoring breathing 1 illustrated comprises a textile support 2 and a plurality of sensors 3 and 4 integrated into the textile support 2 in order to monitor the breathing of a user.

The textile support 2 appears as a clothing item, such as a tee-shirt for example, able to cover the body of the user.

In the embodiment illustrated in FIGS. 1 to 4, the sensors include two breathing sensors 3 and 4.

The breathing sensors 3 and 4 allow detection of the respiratory movement of the thorax cavity and/or of the abdomen of the user who wears the clothing item.

The textile support 2 comprises a tubular portion 21 able to surround the chest of the user.

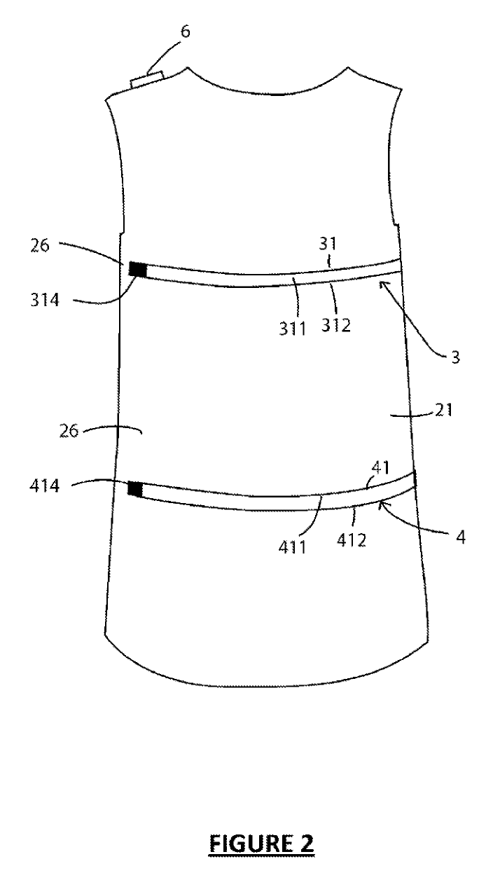

The tubular portion 21 has a neck 24 for letting through the head, a front 25 (visible in FIG. 1) able to cover a belly portion of the user and a back 26 (visible in FIG. 2) capable of covering a portion of the back of the user.

The tubular portion 21 is formed by simultaneous knitting of an electrically insulating, majority ground yarn, and of an elastic yarn.

The majority ground yarn 16 is schematically illustrated in FIG. 10. The majority ground yarn 16 is a synthetic thread, made of a polymer material, such as a polyamide thread (PA) or a polyester thread (PES).

The elastic yarn (not shown) is a yarn made of a thermoplastic elastomer or in a polyurethane derivative, known under the brand of Lycra® (marketed by the company Invista). The elastic yarn may be wrapped (in French “guipé”) or double-wrapped (in French “double-guipé”).

The ground yarn 16 and the elastic yarn are knitted together so that the ground yarn exactly covers the elastic yarn.

Each breathing sensor 3 and 4 extends around the chest of the user in an area located between the top of the sternum and the bottom of the belly.

Each breathing sensor 3 and 4 is formed by knitting an electrically conductive detection yarn.

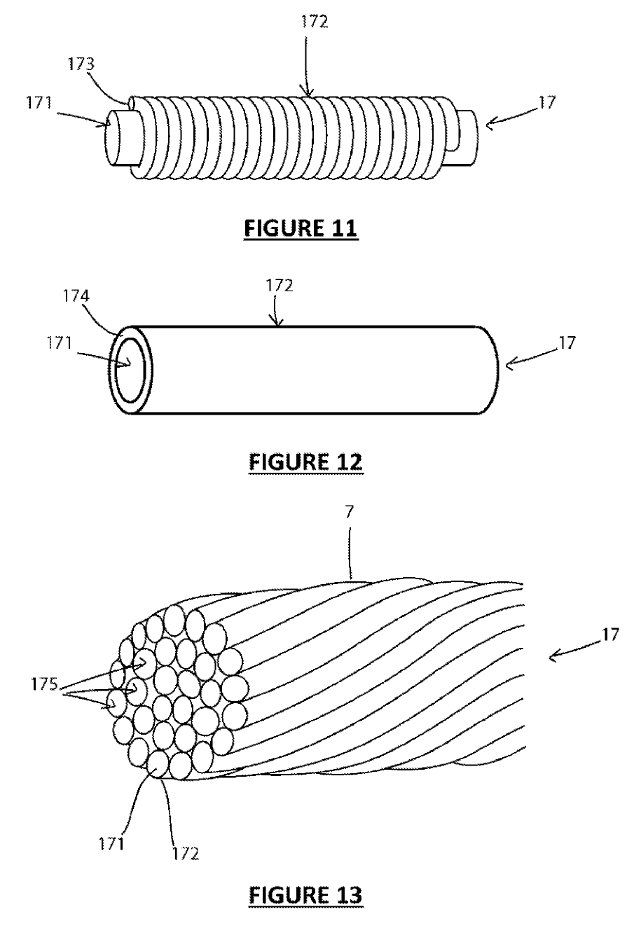

As illustrated in FIGS. 11 to 13, the detection yarn 17 comprises at least one internal core 171 made of an electrically insulating material and an external sheath 172 surrounding the internal core, the external sheath 172 being made of an electrically conducting material. The electrically insulating material of the core 171 may be a polymer, such as polyamide for example.

The conductive material of the external sheath 172 may be a metal, preferably a biocompatible metal, such as silver.

According to a first possibility (illustrated in FIG. 11), the sheath 172 of the detection yarn 17 is formed by wrapping (in French “guipage”) the internal core 171 with a yarn 173 made of a conductive material. In other words, the yarn 173 made of a conductive material is helically wound around the internal core 171.

According to a second possibility (illustrated in FIG. 12), the sheath 172 of the detection yarn is formed by coating the internal core 171 with a layer 174 made of a conductive material. The coating may be achieved by a vacuum deposition technique; by cathode sputtering for example, of the conductive material.

According to a third possibility (illustrated in FIG. 13), the detection yarn 17 consists of a bundle of conductive filaments 175 twisted together. Each conductive filament comprises a core 171 made of an insulating material covered with an external layer 172 made of a conductive material, preferably of silver.

As this is visible in FIGS. 1 to 4, the first breathing sensor 3 forms a first conductive band 31 extending around the chest of the user at the sternum, when the user wears the clothing item. The first conductive band 31 may comprise several branches extending parallel with each other around the chest of the user.

More specifically, in FIGS. 1 to 4, the first conductive band 31 comprises two longitudinal branches 311, 312 extending parallel with each other.

Each longitudinal branch 311, 312 extend along the knitting direction, i.e. parallel to the direction of a row.

Further, each longitudinal branch 311, 312 extends both on the front and on the rear of the clothing item.

The first band 31 also comprises junction portions 313, 314 extending transversely to the knitting direction, and electrically connecting the longitudinal branches 311, 312 with each other at their ends.

The first branch 311 is interrupted at a central axis X of the front of the clothing item (a virtual axis passing through the navel of the user).

The first band 31 forms an electric circuit having two ends 315, 316 located at a distance from each other, on either side of the central axis X. The ends 315 and 316 of the first band 31 are able to be electrically connected to an apparatus for measuring the electric resistance of the first conductive band 31.

Each branch 311, 312 has a width comprised between 1 and 50 rows of stitches.

The second breathing sensor 4 forms a second conductive band 41 extending around the chest of the user at the abdominal muscles.

The second conductive band 41 is similar to the first conductive band 31. The second conductive band 41 comprises two branches 411, 412 extending parallel with each other around the chest of the user and two junction portions 413, 414. The first branch 411 is interrupted at a central axis X. The second conductive band 41 thus also has two ends 415 and 416 located at a distance from each other, on either side of the axis X, and able to be electrically connected to an apparatus for measuring the electric resistance of the second conductive band 41.

The tubular portion 21 and the breathing sensors 3 and 4 are formed by circular knitting in a single operation. The insertion of the conductive yarns is said to be “by means of embroidery” (in French “par le biais de la broderie”).

Thus, the ground yarn 16 (with the elastic yarn) forming the main tubular portion 21 and the detection yarn 17 forming the sensors 3 and 4 are knitted alternately during the knitting operation.

In other words, during the knitting operation, the ground yarn 16 stops being knitted when the detection yarn 17 is knitted. Also, the detection yarn 17 stops being knitted when the ground yarn 16 is knitted.

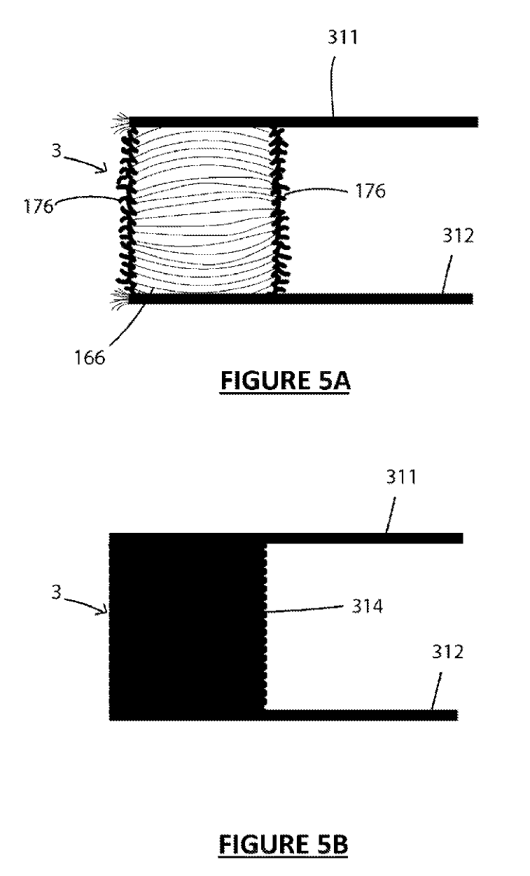

Further, as illustrated in FIGS. 5A and 5B, the detection yarn 17 is cut when the ground yarn 16 is knitted. In FIG. 5A, the cut ends 176 of the detection yarn 17 appear on the back of the clothing item along the edges of the junction portions 313, 314 (also along the edges of the junction portions 413, 414) of the breathing sensors.

On the other hand, the ground yarn 16 is not cut so that non-knitted portions 166 of the ground yarn appear on the back of the clothing item behind the junction portions 313, 314 (also along the edges of the junction portions 413, 414) of the breathing sensors.

Moreover, the device 1 for monitoring breathing comprises one or several pockets attached on the textile support by crimping of a metal part of the type of press buttons, eyelets or rivets, by sewing, welding or thermally bonding. These additional pockets allow the insertion of electronic components into the clothing item, such as a battery or a measuring apparatus for example. In order to limit the displacement of the electronic components with respect to the textile support, the dimensions of each pocket are less than the dimensions of the component which it receives. The insertion of the component into the pocket is possible because of the elastic properties of the textile support.

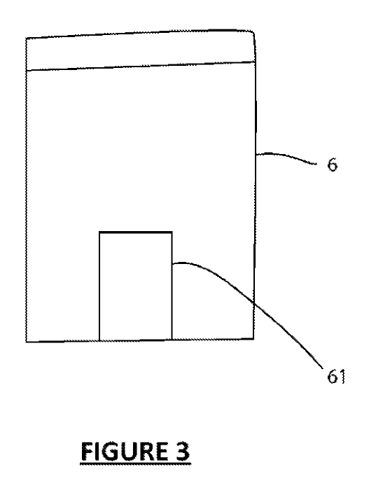

In particular, the device 1 comprises a pocket 6 (visible in FIGS. 1 to 4) and an apparatus for measuring resistance 61 (visible in FIG. 3) accommodated in the pocket 6. The pocket 6 is positioned on the textile support 2 so as to be located on a shoulder of the user when the user wears the clothing item. This position gives the possibility of minimizing the discomfort generated by the presence of the apparatus when the user is lying down. The apparatus for measuring the resistance 61 is able to measure and record the resistance variations of the conductive bands 31 and 41, in order to track the breathing of the user.

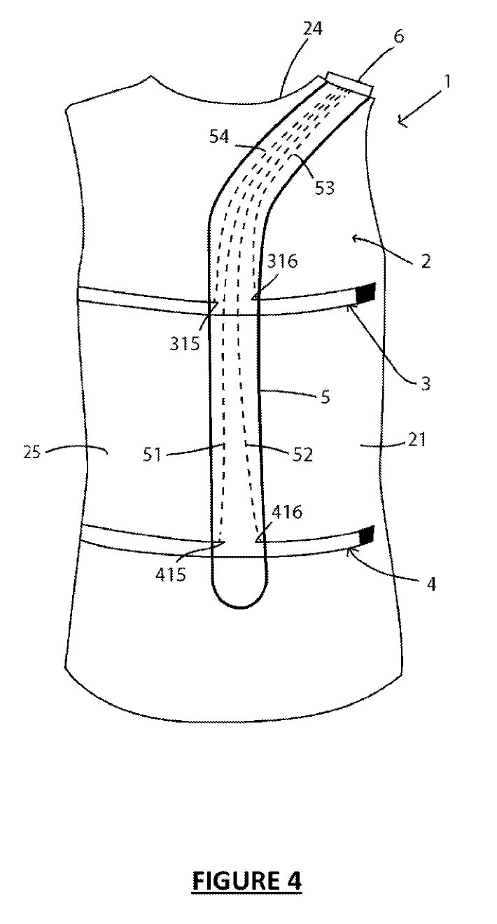

The device 1 for monitoring breathing further comprises a central sheath 5 (visible in FIG. 4) attached onto the tubular portion 21 along the central axis X and electric connecting cables 51 to 54 located inside the sheath 5 so as to connect to each of the sensors to the measuring apparatus 61. The central sheath extends along the central axis X of the clothing item. The central sheath 5 is preferably attached on the back of the device.

The connection of the breathing sensors 3 and 4 is achieved in the following way. An exposed end of a connecting cable 51, 52, 53, 54 is sandwiched between an end 315, 316, 415, 416 to be connected and an added conductive textile part. The textile part is attached by adhesive bonding on the end 315, 316, 415, 416 by means of an adhesive. For example the adhesive used is an adhesive based on polyprocaprolactone (PCL).

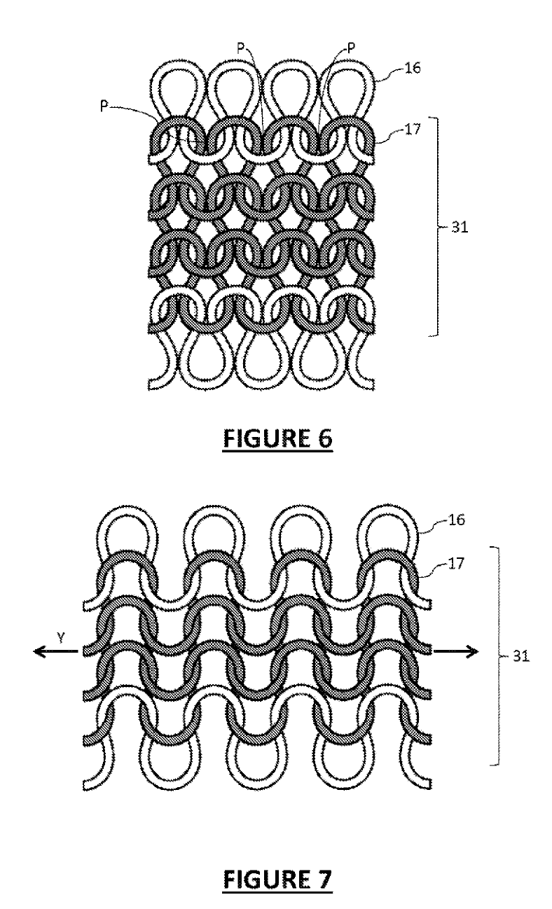

FIGS. 6 and 7 schematically illustrate the structure of the conductive band 31 forming the breathing sensor 3, in the rest condition and in the stretched condition respectively.

The ground yarn 16 is knitted so as to form a plurality of rows.

Also, the detection yarn 17 is knitted so as to form a plurality of rows.

The knitting technique used for the detection yarn 17 is weft-knitting (i.e. the stitches formed by a same continuous yarn are positioned in a same row), preferably with a Jersey base. The same knitting technique may be used for the ground yarn 16.

Each row consists of a plurality of successive stitches. The stitches of a same row form loops alternatively curved in one direction and then in the other, so that the stitches of the row are alternatively interlaced with the stitches of the immediately lower row and with the stitches of the immediately upper row.

As illustrated in FIG. 6, when the conductive band 31 is in the rest condition, the stitches of a same row are in contact with each other in a plurality of contact points P.

As illustrated in FIG. 7, when the conductive band 31 is stretched in a direction Y parallel to the knitting direction (i.e. the direction of a row), the stitches of the detection yarn 17 move away from each other, which reduces the number of contact points P between the stitches.

The separation of the stitches thus causes a reconfiguration of the contact points P within the conductive band 31, which has the effect of modifying the electric resistance of the conductive band 31.

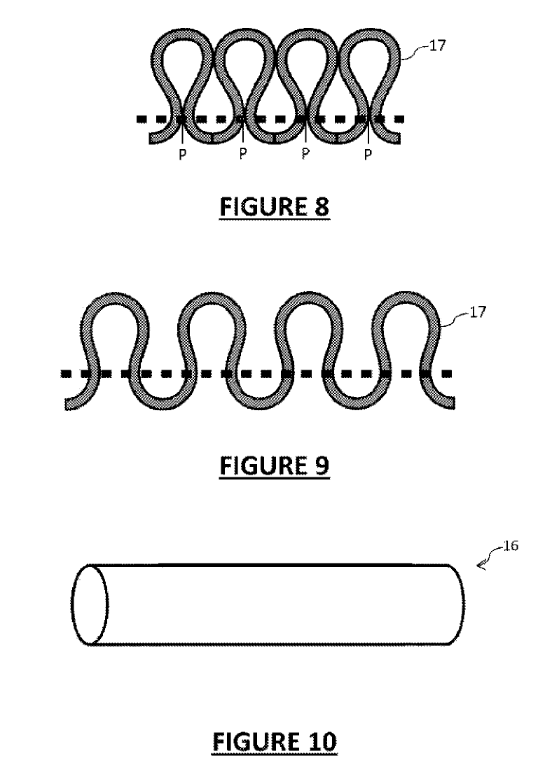

More specifically, as this is illustrated in FIG. 8, when the conductive band 31 is in the rest condition, an electric current may flow along the detection yarn 17 via the contact points P along the line in dotted lines.

On the other hand, as this is illustrated in FIG. 9, when the conductive band 31 is stretched, these contact points disappear which increases the effective electric resistance of the detection yarn 17.

Thus, by measuring the variations in electric resistance of the conductive band 31, it is possible to detect the respiratory movements of the user.

The same principle applies to the conductive band 41.

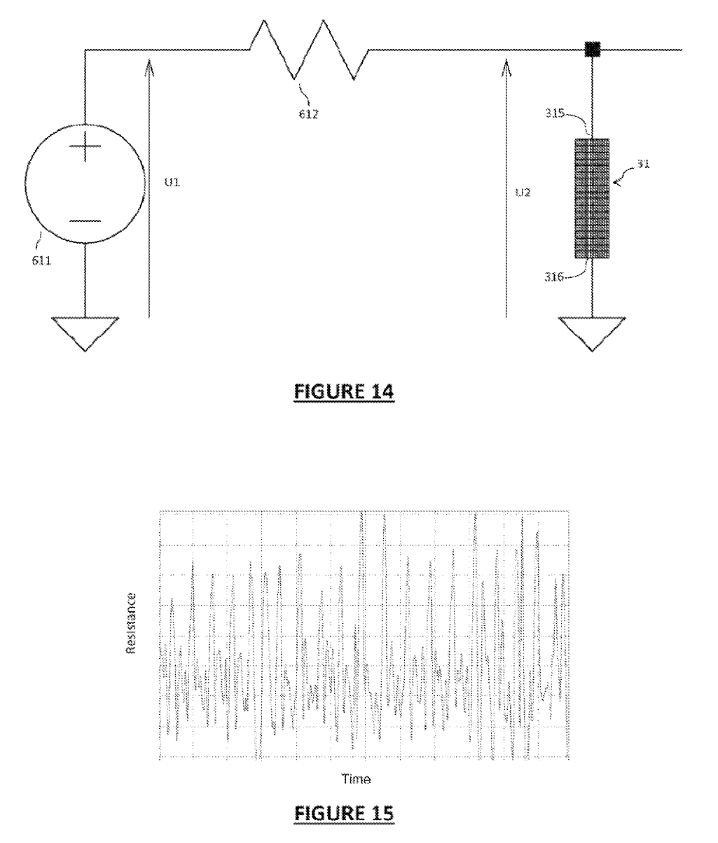

FIG. 14 is a block diagram of an electric circuit of an apparatus 61 for measuring the electric resistance of the conductive band 31.

The apparatus 61 comprises a voltage generator 611, and a first resistor 612. The voltage generator 611 generates a low input voltage U1, of the order of 10 millivolts.

The output voltage U2 generated on the terminals of the conductive band is equal to:

U2=U1*R/(R+R612)

wherein R is the resistance of the conductive band 31, R612 is the resistance value of the resistor 612 and U1 is the input voltage generated by the generator 611.

By measuring the voltage between the ends 315 and 316 of the conductive band 31, it is possible to infer therefrom the resistance R of the conductive band 31.

FIG. 15 is a diagram illustrating the variations of the resistance of the conductive band 31 measured over time when the device 1 is worn by a user who is breathing.

The resistance of the conductive band 31 directly depends on its extension. The measured variations of resistance may be processed in order to monitor breathing parameters, such as the breathing rate of the user or the amplitude of the breathing cycles.

The device 1 may further comprise one or several insulating layers (not shown), positioned between the conductive band(s) 31 and 41, and the skin of the user when the user is covered with the textile support 2. The insulating layer(s) may be formed with an extensible fabric or membrane. The insulating layer(s) give the possibility of electrically insulating the skin of the user from the conductive bands 31 and 41 in which the electric current flows.

1. A device for monitoring the breathing of a user comprising:
a textile support comprising a tubular portion formed by knitting an electrically insulating majority ground yarn, the tubular portion being able to cover the chest of the user,
at least one breathing sensor formed by knitting a detection yarn, the detection yarn forming a plurality of stitches, the detection yarn comprising at least one internal core made of an electrically insulating material and an external sheath surrounding the internal core, the external sheath being made of an electrically conducting material so as to generate electric contacts between the stitches of the detection yarn,
wherein the breathing sensor forms a conductive band having a first end and a second end positioned at a distance from each other, the ends being able to be connected to an apparatus for measuring the electric resistance of the conductive band,
and the conductive band is positioned with respect to the tubular portion so that when the chest of the user is covered with the textile support, the conductive band is alternatively stretched and shrunk because of the breathing of the user, the stretching and the shrinking of the conductive band having the effect of modifying the electric contacts between the stitches of the detection yarn within the conductive band, causing a modification in the electric resistance of the conductive band.

2. The device according to claim 1, wherein the detection yarn comprises at least one core made of a polymeric material, preferably of polyamide.

3. The device according to claim 1, wherein the sheath of the detection yarn is formed by wrapping the internal core with a yarn made of a conductive material, preferably of silver.

4. The device according to claim 1, wherein the sheath of the detection yarn is formed by coating the internal core with a layer made of a conductive material, preferably of silver.

5. The device according to claim 1, wherein the detection yarn consists of several conductive filaments, each conductive filament comprising a core coated with an external layer made of a conductive material, preferably of silver.

6. The device according to claim 5, wherein the coated filaments are twisted together.

7. The device according to claim 1, wherein the tubular portion is formed by knitting the majority ground yarn and an elastic yarn.

8. The device according to claim 1, wherein the conductive band surrounds the chest or a portion of the chest of the user, preferably at the height of the sternum and/or the abdominal muscles, and extends over the belly portion and/or over the back portion of the user.

9. The device according to claim 1, wherein the tubular portion and the breathing sensor are formed by circular knitting in a single operation, the ground yarn and the detection yarn being alternately knitted.

10. The device according to claim 9, wherein, during the knitting, the detection yarn is cut when the ground yarn is knitted.

11. The device according to claim 1, further comprising an insulating layer positioned between the conductive band and the skin of the user when the user is covered with the textile support.

12. The device according to claim 1, further comprising a sheath attached on the tubular portion and connecting cables located inside the sheath for connecting the ends of the conductive band to the apparatus for measuring the electric resistance.
